(19) United States
(12) Patent Application Publication
(10) Pub. No.: US 2024/0148510 A1
(43) Pub. Date: May 9, 2024

(54) ANTI-ROTATION MECHANISM FOR A SACROILIAC SCREW

(71) Applicant: Astura Medical Inc., Iriving, TX (US)

(72) Inventors: Andrew Sprick, Irving, TX (US); Thomas Purcell, Irving, TX (US)

(73) Assignee: Astura Medical Inc., Irving, TX (US)

(21) Appl. No.: 18/378,636

(22) Filed: Oct. 10, 2023

Related U.S. Application Data
(60) Provisional application No. 63/414,816, filed on Oct. 10, 2022.

(51) Int. Cl. A61F 2/30

(52) U.S. Cl. CPC A61F 2/30988; A61F 2002/30433; A61F 2002/30492; A61F 2002/30787; A61F 2002/30858; A61F 2002/30863; A61F 2002/3092; A61F 2002/30995

(57) ABSTRACT

An anti-rotation mechanism for a sacroiliac screw that is designed to prevent screw backout. The sacroiliac screw includes a screw head cap coupled to a screw implant. The anti-rotation mechanism includes geometric features on the screw head cap and/or screw implant that allows rotation in a first direction and prevents rotation in a second or opposite direction. The geometric anti-rotation mechanism for the sacroiliac screw is unique due to the self-contained mechanism within the screw assembly. There are no extra components or process steps required to achieve rotational fixation between the screw body and the screw head cap. The screw allows for the reduction in operating process time compared to other devices that connect the screw head cap directly to the screw body

### CROSS-REFERENCE TO RELATED APPLICATIONS

This application claims the benefit of U.S. Provisional Application No. 63/414,816 filed Oct. 10, 2022, which is incorporated herein by reference.

### FIELD

The present invention relates generally to the field of surgery, and more specifically, to a screw with an anti-rotation mechanism for use in sacroiliac joint fusion surgery.

### BACKGROUND

The sacroiliac (“SI”) joint is a joint located in the pelvis formed by the juncture between the sacrum and the ilium. The SI joint can be a significant contributing factor in many patients having lower back pain. Current competitive Sacroiliac Joint (SI) Fusion screws lack the mechanisms to prevent the screws, or other devices, from retracting or backing out after initial insertion.

Some screws try to prevent screw back out by adding an external washer/screw head cap to the screw implant that is seated above the bone material surface. This allows for secondary teeth features to dig into the exterior bone material to provide additional implant fixation to the primary screw thread fixation. One problem to this is the affixed washers/screw head caps are rotational independent from the primary screw implant. Therefore, if the screw were to unthread from the joint, the screw is free to rotate out and push the secondary fixation cap axially away from the bone surface.

Other screws try to prevent screw back out by affixing a screw head cap to the screw implant, which is seated flush/sub flush to the bone material surface. The cap contains fixation features, such as radial fins to prevent screw retraction after implantation. After screw implant insertion, a set screw is threaded onto the screw head which mechanically locks the implant screw to the screw head cap. Adding the screw which connects the screw head cap to the screw implant body, increases the operation steps and instrumentation required to perform the surgery.

There is a need for an anti-rotation mechanism of the secondary fixation features, used in conjunction with primary fixation features, would prevent a screw from backing out of the joint, after implantation, and prior to proper bone fusion.

### SUMMARY

The present invention is a screw head cap uses secondary bone fixation features in addition to the primary implant screw threads. An anti-rotation mechanism for the screw head cap utilizes a flexible or nitinol pin imbedded in the cap, in conjunction with ratcheting geometric features on the screw. This allows the screw head cap to be permanently fixed to the implant screw and maintain a one direction rotation dependency between the cap and the screw. Rotation in the undesired direction (counterclockwise from the clockwise screw threads) will result in pin interference and not allow the screw head cap or screw to rotate.

### BRIEF DESCRIPTION OF THE DRAWINGS

FIG. 1 is a perspective view showing a one embodiment of a sacroiliac screw.

FIG. 2 is an exploded perspective view of the sacroiliac screw of FIG. 1.

FIG. 3 is enlarged view of the geometric of the sacroiliac screw of FIG. 1.

FIG. 4 is a perspective view showing another embodiment of a sacroiliac screw.

FIG. 5 is a perspective end view of the sacroiliac screw of FIG. 4.

FIG. 6 is an exploded perspective view of the proximal end of the sacroiliac screw of FIG. 4.

FIG. 7 is a perspective view showing another embodiment of a sacroiliac screw.

FIG. 8 is a perspective end view of the sacroiliac screw of FIG. 7 showing internal features.

FIG. 9 perspective view showing another embodiment of a sacroiliac screw.

FIG. 10 is an exploded perspective end view of the sacroiliac screw of FIG. 9.

FIG. 11 is an exploded perspective end view of the proximal end of the sacroiliac screw of FIG. 9.

FIG. 12 is a perspective view of FIG. 9 showing internal features.

FIG. 13 perspective view showing another embodiment of a sacroiliac screw.

FIG. 14 is an exploded perspective end view of the sacroiliac screw of FIG. 13.

FIG. 15 is an exploded perspective end view of the proximal end of the sacroiliac screw of FIG. 13.

### DETAILED DESCRIPTION

The present invention discloses an anti-rotation mechanism for a sacroiliac screw that is designed to prevent screw backout. The sacroiliac screw includes a screw head cap coupled to a screw implant with a retaining ring or split washer. The anti-rotation mechanism includes geometric features on the screw head cap and/or screw implant that allows rotation in a first direction and prevents rotation in a second or opposite direction. The geometric anti-rotation mechanism for the sacroiliac screw is unique due to the self-contained mechanism within the screw assembly. There are no extra components or process steps required to achieve rotational fixation between the screw body and the screw head cap. The screw allows for the reduction in operating process time compared to other devices that connect the screw head cap directly to the screw body.

The present invention also discloses the sacroiliac screw having primary and secondary bone fixation features that may be used in conjunction with the anti-rotation mechanism. The primary bone fixation feature includes screw threads on screw implant and the secondary bone fixation incudes teeth or fins features on the surface of the screw head cap. The anti-rotation mechanism allows for improved bone fixation of the SI Joint Fusion screw. The primary screw thread fixation features will not be able to rotate (and unthread) relative to the secondary fixation teeth. Therefore, the primary and secondary bone fixation features act dependently, rather than independently.

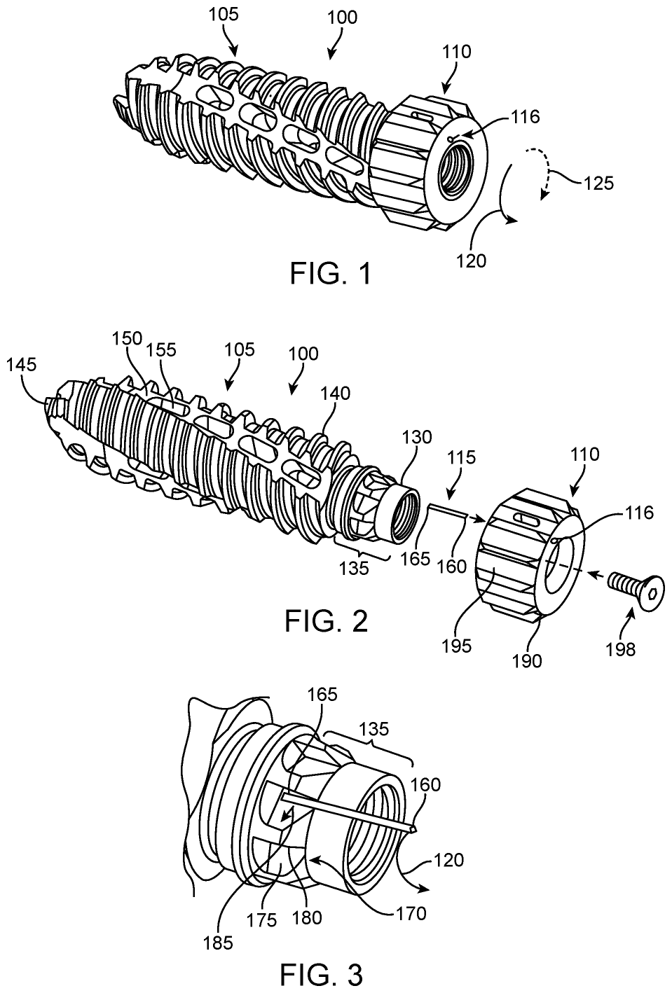

FIG. 1 is a perspective view and FIG. 2 is an exploded perspective view showing one embodiment of a sacroiliac screw 100 having a screw implant 105, a screw head cap 110 and a flexible or nitinol pin 115. The sacroiliac screw 100 also includes an anti-rotation mechanism that utilizes the nitinol pin 115 imbedded in a pin hole 116 in the screw head cap 110, in conjunction with ratcheting geometric features on the screw 105 configured to allow the screw head cap 110 to rotate in a first direction 120 and prevent the screw head cap 110 from rotating in a second or opposite direction 125. This allows the screw head cap 110 to be permanently fixed to the implant screw 105 and maintain a one direction rotation 120 dependency between the screw head cap 110 and the implant screw 105. Rotation in the second or opposite direction 125 (counter-clockwise from the clockwise screw threads) will result in pin interference and not allow the screw head cap 110 or screw implant 105 to rotate. The nitinol pin 115 possesses unique material properties to act as both a spring, and a hard stop, in this application.

The screw implant 105 is a hollow screw implant having a proximal end 130 with geometric features 135, external screw threads 140 extending to a distal tip 145, and bone cutting flutes 150 with openings 155 to receive the cut bone. The screw head cap 110 is rotatably coupled to the proximal end 130 of the screw implant 105. The screw head cap 110 is coupled to the screw implant 105 with a set screw 198.

The nitinol pin 115 includes a proximal end 160 fixedly coupled to the screw head cap 110 and a distal end 155 flexibly engaged with the geometric features 135 on the screw implant 105 to allow the screw head cap 110 to rotate in the first direction 120 and prevent the screw head cap 110 to rotate in the second or opposite direction 125 (counter-clockwise from the clockwise screw threads) will result in pin interference and not allow the screw head cap 110 or screw implant 105 to rotate.

FIG. 3 is enlarged view of the geometric features 135 on the proximal end of the screw implant 105 engaging the nitinol pin 145. The screw head cap 110 has been omitted for clarity of the components. The proximal end 130 of the screw implant 105 includes a plurality of a radial cut outs or pockets 170 having a ramped portion 175 and a flat wall portion 180. When the screw head cap 110 is rotated in the first direction 120, the nitinol pin 115 translates 185 up the ramped portion 175, which flexes the distal end 165 upward or outward radially, until the distal end 165 engages the next radial cut out 170. At this time the distal end 165 drops into the next cut out 170 and the flat wall portion 180 is shaped to engage the nitinol pin 115 and prevent rotation of the screw head cap 110 in the second or reverse direction 125.

The sacroiliac screw 100 also includes a primary bone fixation feature and a secondary bone fixation feature. The primary bone fixation feature is the screw threads 140 on the screw implant 105 and the secondary bone fixation feature is radial fins 190 on the screw head cap 110. A screw head cap 110 is affixed to the screw implant 105, which is seated flush/sub flush to the bone material surface. The radial fins 190 on a radial outer surface 195 of the screw head cap 110 prevent sacroiliac screw 100 retraction after implantation. After sacroiliac screw 100 implantation, the set screw 198 or other fixation device may be threaded onto the threaded proximal end 130 of the screw implant 105 which mechanically locks the screw head cap 110 to the screw implant 105.

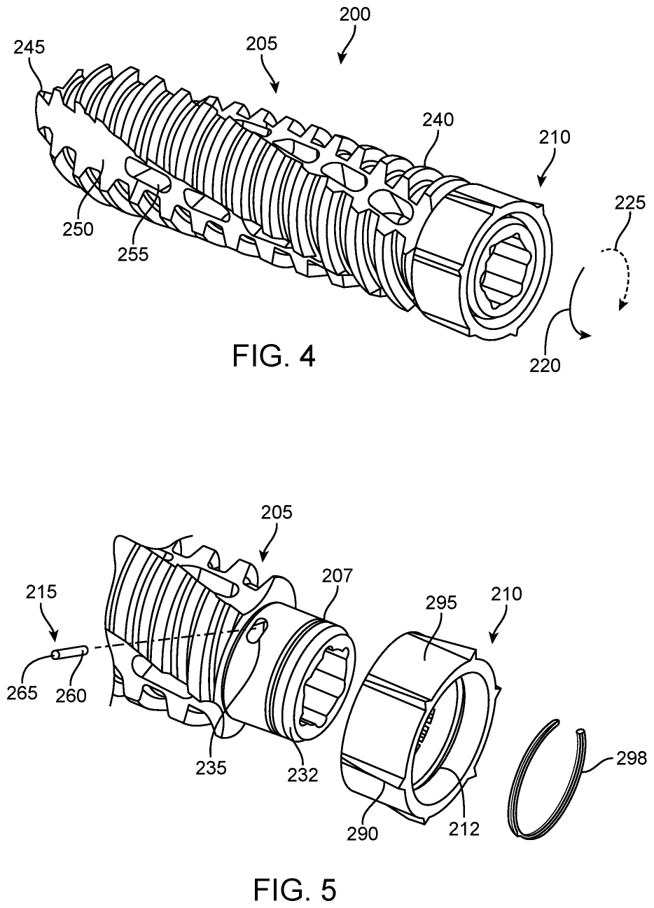

FIG. 4 is a perspective view and FIG. 5 is a perspective end view showing another embodiment of a sacroiliac screw 200 having a screw implant 205, a screw head cap 210 and a flexible or nitinol pin 215. The sacroiliac screw 200 also includes an anti-rotation mechanism that utilizes the nitinol pin 215 imbedded within the screw implant 205, in conjunction with ratcheting geometric features on the screw head cap 210. The nitinol pin 215 location will not have the ability to disassemble once the screw head cap 210 is attached. This allows the screw head cap 210 to maintain a one direction rotation dependency between the screw head cap 210 and the implant screw 205.

The sacroiliac screw 200 includes “engagement geometry” holding the screw head cap 210 to the implant screw 205. The “engagement geometry” includes three components, 1) an external groove 207 on the proximal end 230 of the implant screw 205, 2) an internal groove 212 on the interior surface of the screw head cap 210, and 3) a split washer or retaining ring 298 sized to fit within the external groove 207 and the internal groove 212 to rotatably couple the screw head cap 210 to the implant screw 205.

In one embodiment, the split washer 298 is position within screw head groove 212. As the screw head cap 210 is pushed on the proximal end 230 of the implant screw 205, the split washer 298 contacts a ramped portion 232 on the proximal end 230 and expands outwardly further into the internal groove 212. The internal groove 212 is large enough for the split washer 298 to expand flush with the interior surface of the screw head cap 210 and slide distally on the outer surface of the proximal end 230. Once the split washer 298 reaches the external groove 207, the split washer 298 snaps into the external groove 207, locking the screw head cap 210 on the implant screw 205. In another embodiment, the split washer 298 may be position within the external groove 207.

The screw implant 205 is a hollow screw implant having a proximal end 230 with a pin hole 235 with an elongated entry, and external screw threads 240 extending to a distal tip 245, and bone cutting flutes 250 with openings 255 to receive the cut bone. The nitinol pin 215 includes a first end 260 inserted into the pin hole 235 and a second end 265 extending from the pin hole 235. The second end 265 is configured to flex downward 285 into the elongated entry of the pin hole 235 when force is applied until the second end 265 is flush with a radial surface of the proximal end 230.

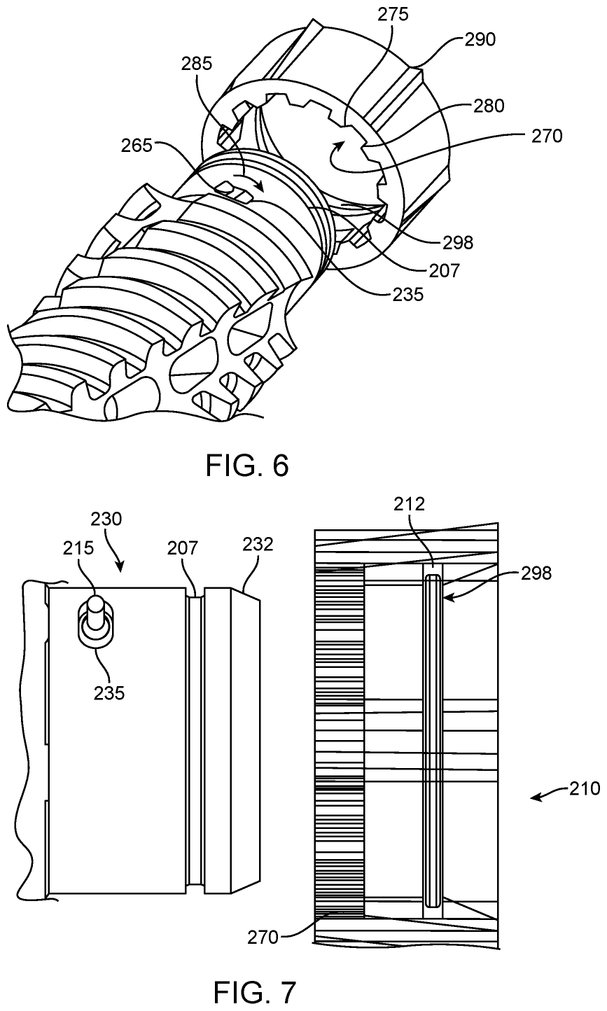

FIG. 6 is an exploded perspective view of the proximal end of the sacroiliac screw 200 showing the geometric features within the screw head cap 210 that are configured to engage the second end 265 of the nitinol pin 215. The interior portion of the screw head cap 210 includes a plurality of a radial cut outs or pockets 270 having a ramped portion 275 and a flat wall portion 280 and the split washer 298 position within screw head groove 212.

When the screw head cap 210 is rotated in the first direction 220, the second end 265 of the nitinol pin 215 contacts the ramped portion 275 and flexes downward 285 into the elongated entry of the pin hole until the second end 265 engages the next radial cut out 270. At this time the second end 265 flexes out and drops into the next cut out 270 and the flat wall portion 280 is shaped to engage the second end 265 of the nitinol pin 215 and prevent the screw head cap 210 to rotate in the second or opposite direction 225 (counter-clockwise from the clockwise screw threads) will result in pin interference and not allow the screw head cap 210 or screw implant 205 to rotate.

The sacroiliac screw 200 also includes a primary bone fixation feature and a secondary bone fixation feature. The primary bone fixation feature is the screw threads 240 on the screw implant 205 and the secondary bone fixation feature is radial fins 290 on the screw head cap 210. A screw head cap 210 is affixed to the screw implant 205, which is seated flush/sub flush to the bone material surface. The radial fins 290 on a radial outer surface 295 of the screw head cap 210 prevent screw 200 retraction after implantation. After screw implantation, a set screw or other fixation device may be threaded onto the proximal end 230 of the screw implant 205 which mechanically locks the screw head cap 210 to the screw implant 205.

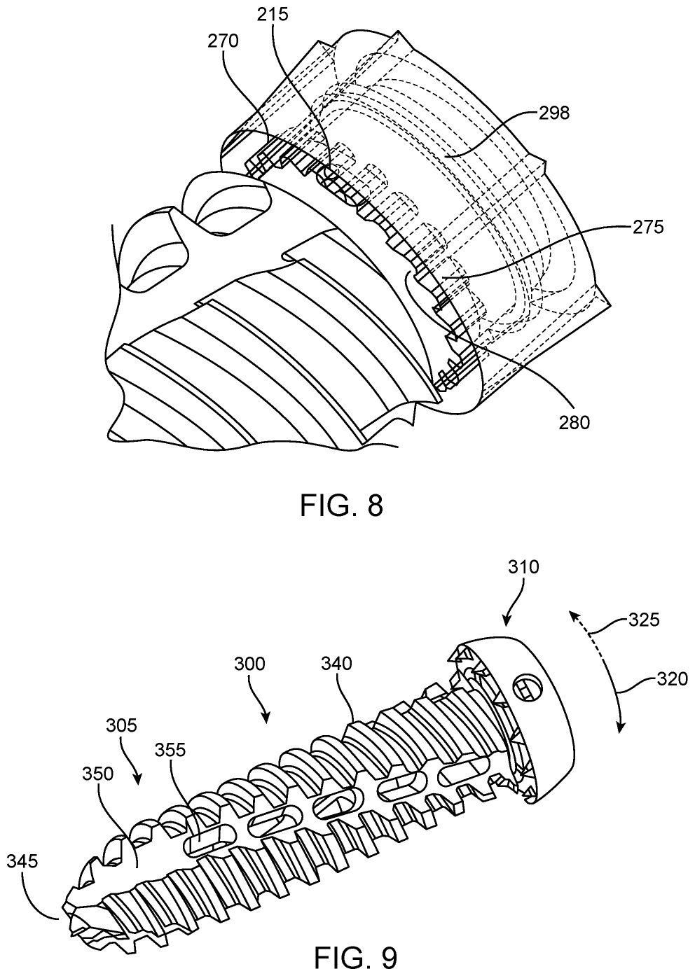

FIG. 7 is a side view and FIG. 8 is a perspective proximal view showing the proximal end 230 and screw head cap 210, which is opaque to show internal features. The nitinol pin 215 is positioned within the pin hole 235 with the proximal end 265 extended above the radial surface and configured to engage the ramped portions 275 and flat wall portions 280 in the radial cut outs or pockets 270. The ramped portion 232 on the proximal end 230 configured to expand the split washer 298 and external groove 207 configured to receive the split washer to couple the screw head cap 210 to the screw implant 205.

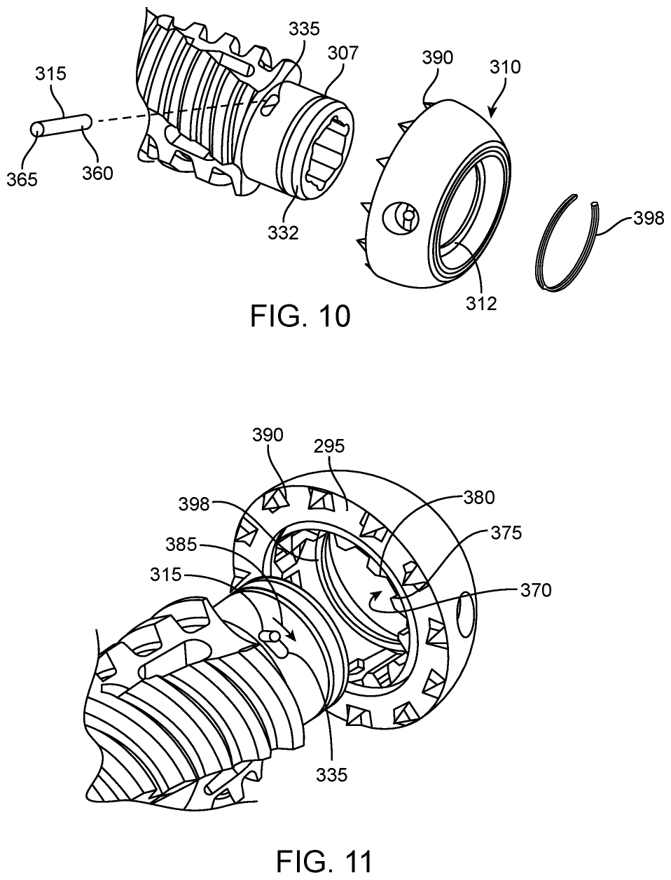

FIG. 9 is a perspective view and FIG. 10 is an exploded perspective view showing another embodiment of a sacroiliac screw 300 having a screw implant 305, a screw head cap 310 and a flexible or nitinol pin 315. The sacroiliac screw 300 also includes an anti-rotation mechanism that utilizes the nitinol pin 315 imbedded within the screw implant 305, in conjunction with ratcheting geometric features on the screw head cap 310. The nitinol pin 315 location will not have the ability to disassemble, once the screw head cap 310 is attached. This allows the screw head cap 310 to maintain a one direction rotation dependency between the screw head cap 310 and the implant screw 305.

The sacroiliac screw 300 includes “engagement geometry” holding the screw head cap 310 to the implant screw 305. The “engagement geometry” includes three components, 1) an external groove 307 on the proximal end 330 of the implant screw 305, 2) an internal groove 312 on the interior surface of the screw head cap 310, and 3) a split washer or retaining ring 398 sized to fit within the external groove 307 and the internal groove 312 to rotatably couple the screw head cap 310 to the implant screw 305.

The screw implant 305 is a hollow screw implant having a proximal end 330 with a pin hole 335 with an elongated entry, and external screw threads 340 extending to a distal tip 345, and bone cutting flutes 350 with openings 355 to receive the cut bone. The nitinol pin 315 includes a first end 360 inserted into the pin hole 335 and a second end 365 extending from the pin hole 335. The second end 365 is configured to flex downward 385 into the elongated entry of the pin hole 335 when force is applied until the second end 365 is flush with a radial surface of the proximal end 330.

FIG. 11 is an exploded perspective view of the proximal end of the sacroiliac screw 300 showing the geometric features within the screw head cap 310 that are configured to engage the second end 365 of the nitinol pin 315. The interior portion of the screw head cap 310 includes a plurality of a radial cut outs or pockets 370 having a ramped portion 375 and a flat wall portion 380. When the screw head cap 310 is rotated in the first direction 320, the second end 365 of the nitinol pin 315 contacts the ramped portion 375 and flexes downward 385 into the elongated entry of the pin hole 335 until the second end 365 engages the next radial cut out 370. At this time the second end 365 flexes out and drops into the next cut out 370 and the flat wall portion 380 is shaped to engage the second end 365 of the nitinol pin 315 and prevent the screw head cap 310 to rotate in the second or opposite direction 325 (counter-clockwise from the clockwise screw threads) will result in pin interference and not allow the screw head cap 310 or screw implant 305 to rotate.

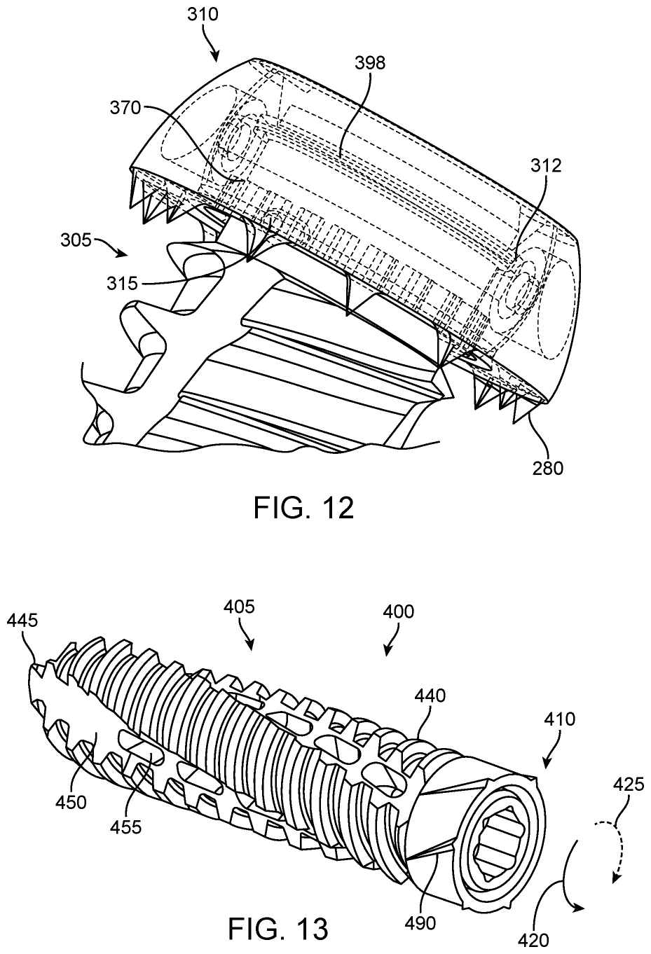

FIG. 12 is a perspective view showing the proximal end 330 and screw head cap 310, which is opaque to show internal features. The nitinol pin 315 is positioned within the pin hole 335 with the proximal end 365 extended above the radial surface and configured to engage the ramped portions 375 and flat wall portions 380 in the radial cut outs or pockets 370. The ramped portion 332 on the proximal end 330 is configured to expand the split washer 398 and external groove 307 configured to receive the split washer to couple the screw head cap 310 to the screw implant 305.

The sacroiliac screw 300 also includes a primary bone fixation feature and a secondary bone fixation feature. The primary bone fixation feature is the screw threads 340 on the screw implant 305 and the secondary bone fixation feature is teeth 390 on a distal surface 395 of the screw head cap 310. A screw head cap or other fixation device may be affixed to the screw implant 305 and the teeth 390 engage the bone material surface. The teeth 290 of the screw head cap 310 prevent screw 300 retraction after implantation.

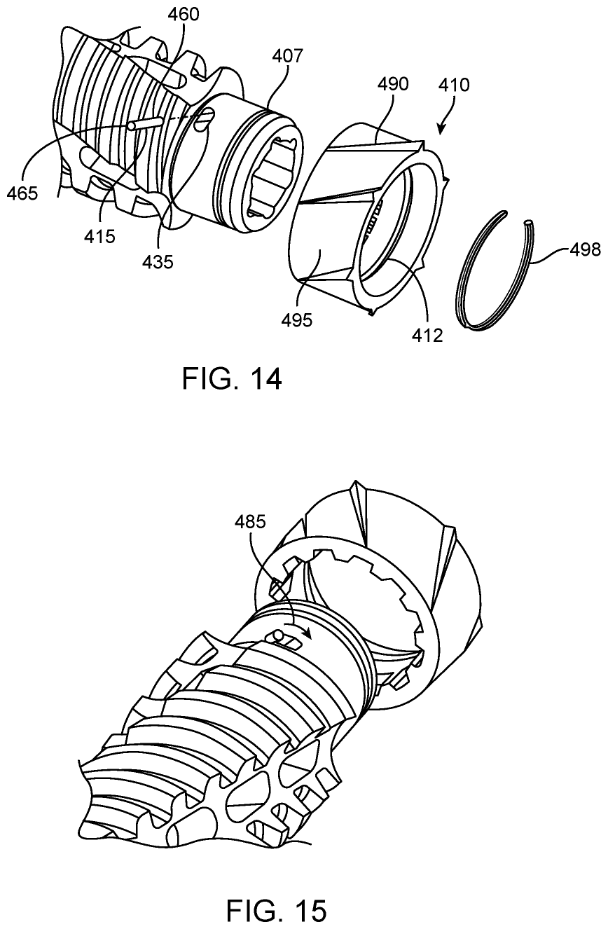

FIG. 13 is a perspective view and FIG. 14 is a perspective end view showing another embodiment of a sacroiliac screw 400 that is similar to sacroiliac screw 200 except the radial fins 490 are angled in a counterclockwise direction. The sacroiliac screw 400 includes a screw implant 405, a screw head cap 410 and a flexible or nitinol pin 415. The sacroiliac screw 400 also includes an anti-rotation mechanism that utilizes the nitinol pin 415 imbedded within the screw implant 405, in conjunction with ratcheting geometric features on the screw head cap 410. The nitinol pin 415 location will not have the ability to disassemble, once the screw head cap 410 is attached. This allows the screw head cap 410 to maintain a one direction rotation dependency between the screw head cap 410 and the implant screw 405.

The sacroiliac screw 400 includes “engagement geometry” holding the screw head cap 410 to the implant screw 405. The “engagement geometry” includes three components, 1) an external groove 407 on the proximal end 430 of the implant screw 405, 2) an internal groove 412 on the interior surface of the screw head cap 410, and 3) a split washer or retaining ring 498 sized to fit within the external groove 407 and the internal groove 412 to rotatably couple the screw head cap 410 to the implant screw 405.

In one embodiment, the split washer 498 is position within screw head groove 412. As the screw head cap 410 is pushed on the proximal end 430 of the implant screw 405, the split washer 498 contacts a ramped portion 432 on the proximal end 430 and expands outwardly further into the internal groove 412. The internal groove 412 is large enough for the split washer 498 to expand flush with the interior surface of the screw head cap 410 and slide distally on the outer surface of the proximal end 430. Once the split washer 498 reaches the external groove 407, the split washer 498 snaps into the external groove 407, locking the screw head cap 410 on the implant screw 405. In another embodiment, the split washer 498 may be position within the external groove 407.

The screw implant 405 is a hollow screw implant having a proximal end 430 with a pin hole 435 with an elongated entry, and external screw threads 440 extending to a distal tip 445, and bone cutting flutes 450 with openings 455 to receive the cut bone. The nitinol pin 415 includes a first end 460 inserted into the pin hole 435 and a second end 465 extending from the pin hole 435. The second end 465 is configured to flex downward 485 into the elongated entry of the pin hole 435 when force is applied until the second end 465 is flush with a radial surface of the proximal end 430.

FIG. 15 is an exploded perspective view of the proximal end of the sacroiliac screw 400 showing the geometric features within the screw head cap 410 that are configured to engage the second end 465 of the nitinol pin 415. The interior portion of the screw head cap 410 includes a plurality of a radial cut outs or pockets 470 having a ramped portion 475 and a flat wall portion 480 and the split washer 498 position within screw head groove 412.

When the screw head cap 410 is rotated in the first direction 420, the second end 465 of the nitinol pin 415 contacts the ramped portion 475 and flexes downward 485 into the elongated entry of the pin hole until the second end 465 engages the next radial cut out 470. At this time the second end 465 flexes out and drops into the next cut out 470 and the flat wall portion 480 is shaped to engage the second end 465 of the nitinol pin 415 and prevent the screw head cap 410 to rotate in the second or opposite direction 425 (counter-clockwise from the clockwise screw threads) will result in pin interference and not allow the screw head cap 410 or screw implant 405 to rotate.

The sacroiliac screw 400 also includes a primary bone fixation feature and a secondary bone fixation feature. The primary bone fixation feature is the screw threads 440 on the screw implant 405 and the secondary bone fixation feature are angled radial fins 490 on the screw head cap 410. In the embodiment shown, the angled radial fins 490 are in a counterclockwise direction but may be angled in other directions or may be curved radial fins. A screw head cap 410 is affixed to the screw implant 405, which is seated flush/sub flush to the bone material surface. The angled radial fins 490 on a radial outer surface 495 of the screw head cap 410 prevent screw 400 retraction after implantation. After screw implantation, a set screw or other fixation device may be threaded onto the proximal end 430 of the screw implant 405 which mechanically locks the screw head cap 410 to the screw implant 405.

While the present invention is described in relation to a sacroiliac screw, the invention is not limited to sacroiliac screws. Other bones screws may also utilize the invention and embodiments described herein.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to persons skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. A sacroiliac screw comprising:
a screw implant having a proximal end with ratcheting geometric features;
a screw head cap rotatably coupled to the proximal end of the screw implant; and
a flexible pin having a first end fixedly coupled with the screw head cap and a second end flexibly coupled with the ratcheting geometric features, the flexible pin is configured to allow screw head cap to rotate in a first direction and prevent screw head cap to rotate in a second direction.

2. The sacroiliac screw of claim 1, wherein the ratcheting geometric features includes a plurality of radial cutouts having a ramped portion and a flat wall portion.

3. The sacroiliac screw of claim 2, wherein when the head cap is rotated in the first direction, the flexible pin is configured to flex radially and translate up the ramped portion of a first radial cutout until the flexible pin engages and drops into a second radial cutout.

4. The sacroiliac screw of claim 2, wherein when the screw head cap is rotated in a second or reverse direction, the flexible pin is configured to engage the flat wall portion of a radial cutout and prevent of the screw head cap in the second direction.

5. The sacroiliac screw of claim 1, wherein the screw head cap includes a pin hole configured to hold the proximal end of the flexible pin.

6. The sacroiliac screw of claim 1, wherein the screw implant includes a primary bone fixation feature and the screw head cap includes a secondary bone fixation feature.

7. The sacroiliac screw of claim 6, wherein the primary bone fixation includes external screw threads on the screw implant extending distally from the ratcheting geometric features to a tip, and the secondary bone fixation feature includes radial fins on an outer surface of the screw head cap.

8. The sacroiliac screw of claim 1, wherein the radial fins are angled in the opposite direction of the external screw threads.

9. A sacroiliac screw comprising:
a screw implant with a proximal end having an outer surface with an outer groove, and a distal end having external screw threads extending distally from the outer surface to a distal tip;
a screw head cap having an inner surface with an inner groove and ratcheting geometric features configured to slide on the outer surface of the screw implant, and an outer surface with radial fins;
a split washer configured to fit within the external groove and internal groove to rotatably couple the screw head cap with the screw implant; and
a flexible pin having a first end fixedly coupled with the outer surface of the screw implant and a second end flexibly coupled with the ratcheting geometric features of the screw head cap;
wherein with the ratcheting geometric features and flexible pin allow rotation of the screw head cap in a first direction and prevent rotation of the screw head cap in a second direction.

10. The sacroiliac screw of claim 9, wherein the radial fins in conjunction with the external screw threads prevent the screw from backing out after implantation.

11. The sacroiliac screw of claim 10, wherein the radial fins and external screw threads are angled in different directions prevent back out.

12. The sacroiliac screw of claim 11, wherein the external screw threads are angled in a clockwise direction and the radial fins angled in a counterclockwise direction.

13. The sacroiliac screw of claim 9, wherein the ratcheting geometric features includes a plurality of radial cutouts having a ramped portion and a flat wall portion.

14. The sacroiliac screw of claim 13, wherein when the head cap is rotated in the first direction, the flexible pin is configured to flex radially and translate up the ramped portion of a first radial cutout until the flexible pin engages and drops into a second radial cutout.

15. The sacroiliac screw of claim 13, wherein when the screw head cap is rotated in a second or reverse direction, the flexible pin is configured to engage the flat wall portion of a radial cutout and prevent of the screw head cap in the second direction.

16. A sacroiliac screw comprising:
a screw implant having external screw threads on a distal end and an external groove on a proximal end;
a screw head cap having an inner surface configured to rotatably couple with the proximal end of the screw implant and an outer surface with radial fins, the inner surface having ratcheting geometric features and an internal groove;
a split washer configured to fit within the external groove and internal groove to prevent the screw head cap from uncoupling with the screw implant; and
a flexible pin having a first end fixedly couple with the screw implant and a second end flexible coupled with ratcheting geometric features to allow rotation of the screw head cap in a first direction and prevent rotation in a second, opposite direction.

17. The sacroiliac screw of claim 9, wherein the radial fins and external screw threads are angled in different directions prevent back out.

18. The sacroiliac screw of claim 17, wherein the external screw threads are angled in a clockwise direction and the radial fins angled in a counterclockwise direction.

19. The sacroiliac screw of claim 16, wherein the ratcheting geometric features includes a plurality of radial cutouts having a ramped portion and a flat wall portion, when the head c.

20. The sacroiliac screw of claim 19, wherein when the screw head cap is rotated in the first direction the flexible pin engages the ramped portion of the radial cutout to allow rotation in the first direction, and when the screw head cap is rotated in the second direction the flexible pin engages the flat wall portion of the radial cutout to prevent rotation of the screw head cap in the second direction.
